(19) United States
(12) Patent Application Publication
(10) Pub. No.: US 2024/0352126 A1
(43) Pub. Date: Oct. 24, 2024

(54) A PHARMACEUTICAL FORMULATION OF IMMUNE CHECK POINT INHIBITORS

(71) Applicant: DR. REDDY’S LABORATORIES LIMITED, Hyderabad (IN)

(72) Inventors: Murali JAYARAMAN, Kancheepuram (Distt) (IN); Saisharan K GOUD, Kamareddy (Distt) (IN); Sunil Ashok NANKAR, Jalgaon (Distt) (IN); Maya NANATH, Palakkad (IN); Lovisha AGGARWAL, New Delhi (IN); Puja SARKAR, Chennai (IN); Mahesh INGALE, Solapur (IN); Reinhard JACOB, Idukki (IN); Abirami SURESH, Namakkal (IN); R. L. Shri Ranga PRIYA, Tiruchirapalli (IN); Suman LABALA, Srikakulam (IN)

(21) Appl. No.: 18/688,170

(22) PCT Filed: Sep. 2, 2022
(86) PCT No.: PCT/IN2022/050790

§ 371 Date: Feb. 29, 2024

(30) Foreign Application Priority Data
Sep. 3, 2021 (IN) 202141040074

(51) Int. Cl. C07K 16/28; A61K 9/08; A61K 39/00; A61K 47/12; A61K 47/18; A61K 47/26

(52) U.S. Cl. CPC C07K 16/2818; A61K 9/08; A61K 47/12; A61K 47/183; A61K 47/26; A61K 2039/505; C07K 2317/21; C07K 2317/24; C07K 2317/94

(57) ABSTRACT

The present invention relates to pharmaceutical formulations of antibodies and antigen-binding fragments against human programmed death receptor-1 (PD-1)/programmed death receptor Ligand 1 (PD-L1), and method for preparing the same. The disclosed formulations are free of chelators and stabilizes anti-PD1/anti-PD L1 antibody from lower to higher concentrations rendering it suitable for different modes of administration (subcutaneous/intravenous).

### FIELD OF THE INVENTION

The present invention relates to the field of therapeutic antibody formulations. More specifically, the present invention relates to the field of stable formulations of antibodies and antigen-binding fragments against human programmed death receptor-1 (PD-1)/programmed death receptor Ligand 1 (PD-L1), and method for preparing the same.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc. All of these stress factors could result in the loss of biological activity of a therapeutic protein/antibody.

Hence, it is necessary to formulate an antibody in an appropriate buffer and also the selection of excipients also impact stability of the antibody in a formulation. The stability of an antibody in a liquid formulation depends not only on the kinds of excipients used in the formulation, but also on the amounts and proportions of the excipients relative to one another. Apart from these, other factors such as viscosity of the formulation, visual appearance must also be taken care while preparing any formulation.

Antibodies which binds to the human programmed death-1 protein (PD-1) or human programmed death ligand-1 protein (PDL-1) are few of the examples of therapeutic antibodies and have gained a lot of importance due to their broad spectrum in treating various oncological disorders.

Although anti-PD-1/PD-L1 antibodies are known, there remains a need in the art for novel pharmaceutical formulations comprising anti-PD-1 antibodies that are sufficiently stable and suitable for administration to human patients.

### SUMMARY

The present invention discloses a pharmaceutical formulation of an anti-PD-1/PD-L1 antibody.

The pharmaceutical formulation of the present invention discloses an anti-PD1/PD-L1 antibody or an antigen-binding fragment thereof, wherein the formulation comprises anti-PD1/PD-L1 antibody, a buffer having pH of 4.5 to 6.5 wherein the formulation is free of chelator/chelating agent. The formulation optionally includes one or more pharmaceutically acceptable stabilizers.

In particular, the disclosed formulations of the invention stabilizes anti-PD1/PD-L1 antibody from lower to higher concentration, from about 10 mg/ml to about 200 mg/ml rendering it suitable for different routes of administration.

In one aspect, the invention discloses a method of controlling particle formation in an anti-PD-1/PD-L1 antibody composition wherein the method comprises addition of succinate buffer or acetate buffer or citrate buffer or histidine buffer, or it's derivatives or salts or combinations thereof, to the antibody composition, wherein the obtained antibody composition is free of any chelator/chelating agent. The said buffer compositions can be added during pre-formulation and/or at formulation stage of the antibody production.

Further, the invention discloses a method of reducing charge variants, deamidation, and/or aggregation of an anti-PD-1/PD-L1 antibody in its composition, wherein the method comprises addition of succinate buffer or acetate buffer or citrate buffer or histidine buffer or it's derivatives or salts or combination thereof to the antibody composition, wherein the obtained antibody composition is free of a chelator/chelating agent. The said buffer composition can be added during pre-formulation and/or at formulation stage of the antibody production.

In addition, the invention discloses a method of controlling opalescence of an anti-PD1/anti-PDL1 antibody composition in its composition, wherein the method comprises addition of succinate buffer or acetate buffer or citrate buffer or histidine buffer or it's derivatives or salts or combination thereof to the antibody composition, wherein the obtained antibody composition is free of a chelator/chelating agent. The said buffer composition can be added during pre-formulation and/or at formulation stage of the antibody production to maintain the antibody in soluble form in the composition, thereby maintaining opalescence.

The invention also discloses a method to impart colloidal stability to an anti-PD1/anti-PDL1 antibody wherein the method comprises formulating the anti-PD1/PD-L1 antibody in a buffer composition comprising succinate buffer or acetate buffer or citrate buffer or histidine buffer or it's derivative or salts or combination thereof.

The pH of the formulation of the present invention is in the range from about pH 4.5 to about pH 6.5.

The disclosed formulations of the invention exhibit stability under at least one of the following accelerated conditions that includes a temperature ranging from 25° C. to 40° C. and for a period of time ranging from 1 day to 28 days/4 weeks. The antibody in the said formulation is stable and maintains 98% or more (≥98%) of monomeric content of the antibody in the formulation even after storage for two weeks at 40° C.

### DETAILED DESCRIPTION OF THE INVENTION

### Definitions

The term “about” used herein would mean and include a variation of upto 20% from the particular value.

The term “antibody” as used herein encompasses whole antibodies or any antigen binding fragment (i.e., “antigen-binding portion”) or fusion protein thereof.

The term “buffer” used herein refers to an agent which resists to any change in pH of a solution, near a chosen value, up on addition of acid or base. The buffer herein includes buffering agents, or its' derivative, or salts and combination thereof.

The term “stable” formulation refers to the formulation wherein the antibody therein retains its physical stability and/or chemical stability and/or biological activity.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Negatively charged molecules bind to anion exchange resins while positively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, salts, surfactants, and derivatives and combination thereof. The at least one stabilizer in a pharmaceutical formulation of the present invention can be a saccharide or an amino acid.

The term sugar/s as used herein includes organic compounds having general formula of all carbohydrates of the general formula Cn(H₂O)n. Sugars can be referred to monosaccharides, disaccharides, and polysaccharides. Examples of sugars include, but are not limited to, sucrose, trehalose, glucose, dextrose, raffinose and others.

The term “polyol” or “sugar alcohol” as used herein includes an organic compound containing multiple hydroxyl groups. Examples of polyols include mannitol, sorbitol, xylitol etc.,

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

Examples of salts include, but not limited to, sodium chloride, potassium chloride, magnesium chloride, sodium thiocyanate, ammonium thiocyanate, ammonium sulfate, ammonium chloride, calcium chloride, zinc chloride and/or sodium acetate.

The term “opalescence” or “opalescent appearance” refers to the degree of turbidity detected in a solution, e.g., a protein preparation, as a function of the concentration of one or more of the components in the solution, e.g., protein and/or salt concentration. The degree of turbidity can be calculated by reference to a standard curve generated using suspensions of known turbidity. Reference standards for determining the degree of turbidity for pharmaceutical compositions can be based on the United States Pharmacopeia or European Pharmacopeia criteria. Here, in this invention to measure opalescence, first Formazine solution has been prepared by mixing equal volumes of a hydrazine sulfate solution and hexamethylenetetramine solution and then diluted to prepare various reference opalescence standards. The opalescence standards includes ROS-I, ROS-II, ROS-III and ROS-IV.

Nephelometry is a turbidometric method used to detect the presence of soluble aggregates or to indicate opalescence. The output is listed in terms of nephelometric turbidity units (NTUs).

“Pre-formulation steps” refers to any or multiple steps performed before formulating the protein into a therapeutic product. Examples of such steps include, chromatography, filtration, (ultrafiltration, sterile filtration, nano filtration, diafiltration, tangential flow filtration, depth filtration), or any other steps performed to concentrate the protein or to exchange the buffer to a different/suitable buffer. The filtration steps mentioned herein may be performed in a tangential flow filtration mode.

“Formulation steps” refers to steps which are followed after the downstream chromatographic and filtration steps, to prepare drug product from drug substance obtained from the pre-formulation steps.

The term “chelator or a chelating agent” refers to a compound which can form at least one bond with a metal atom. A chelating agent is typically a multidentate ligand that can be used in compositions as a stabilizer to complex with species, which might otherwise promote instability. Exemplary agents chelating include aminopolycarboxylic acids, hydroxyaminocarboxylic acids, N-substituted glycines, 2-(2-amino-2-oxoethyl) aminoethane sulfonic acid (BES), deferoxamine (DEF), niacinamide, desoxycholates, ethylenediaminetetraacetic acid (EDTA), diethylenetriaminepentaacetic acid (DTPA), nitrilotriacetic acid (NTA), N-2-acetamido-2-iminodiacetic acid (ADA), bis(aminoethyl) glycolether, N,N,N′,N′-tetraacetic acid (EGTA), trans-diaminocyclohexane tetraacetic acid (DCTA), N-hydroxyethyliminodiacetic acid (HIMDA), N,N-bis-hydroxyethylglycine (bicine), N-(trishydroxymethylmethyl) glycine (tricine), glycylglycine, sodium desoxycholate, ethylenediamine; propylenediamine; diethylenetriamine; triethylenetetraamine (trien), ethylenediaminetetraaceto EDTA; disodium EDTA, EDTA, calcium EDTA oxalic acid and malate.

The term “antioxidant” mentioned herein refers to an agent that inhibits the oxidation of other molecules and is not part of buffer component. Examples of antioxidants herein include methionine, lipoic acid, uric acid, glutathione, tocopherol, carotene, lycopene, cysteine, phosphonate compounds, e.g., etidronic acid, desferoxamine and malate.

### DETAILED DESCRIPTION OF THE EMBODIMENTS

The present invention discloses pharmaceutical formulations of an anti-PD1/anti-PDL1 antibody. Some of the approved anti-PD1 antibody formulations comprises chelating agent as one of the excipients. However, use of chelating agent include its own consequences that may not be beneficial always. For example, addition of chelating agent such as EDTA to a monoclonal antibody formulation leads to enhanced Fe2+ or Fe3+-induced clipping in the monoclonal antibodies. The enhanced catalytic activity is likely because of both acceleration of metal leaching from the container in the presence of a chelating agent and an active state of the metal under chelated state. The present invention provides an improved antibody formulation addressing this problem.

In an embodiment, the invention discloses a pharmaceutical formulation of an antibody against human programmed cell death protein 1 (PD-programmed death receptor Ligand 1 (PDL1) comprising:
(i) an anti-PD-1/anti-PDL1 antibody and
(ii) a buffer having pH of about 4.5 to about 6.5, and,

wherein the formulation is devoid of a chelator or a chelating agent.

In another embodiment, the invention discloses a pharmaceutical formulation of an antibody against human programmed cell death protein 1 (PD-1)/programmed death receptor Ligand 1 (PDL1) comprising:
(i) an anti-PD-1/anti-PDL1 antibody,
(ii) a buffer having pH of about 4.5 to about 6.5,
(iii) one or more stabilizers;
(iv) a surfactant; and,

wherein the formulation is free of chelator or chelating agent.

In any of the above said embodiments, the buffer mentioned in the formulation is an organic buffer or an inorganic buffer, and/or it's salts or combinations thereof.

In the above mentioned embodiment of the invention, the said organic buffer is a succinate buffer or an acetate buffer or a citrate buffer or a histidine buffer and the inorganic buffer mentioned in the formulation is a phosphate buffer.

In an embodiment, the invention discloses a method of imparting colloidal stability to an anti-PD1/PD L1 antibody, in an anti-PD1/PDL1 antibody composition, wherein the method involves addition of a buffer composition comprising a succinate buffer or acetate buffer or citrate buffer or histidine buffer, or it's derivatives or salts or combinations thereof, to the antibody composition, wherein the said buffer composition does not contain any chelator or chelating agent. The said buffer composition is added at the pre-formulation and/or at the formulation steps of the antibody production.

In an embodiment, the invention discloses a pharmaceutical formulation of an antibody against human programmed cell death protein 1 (PD-programmed death receptor Ligand 1 (PDL1) comprising:
(i) an anti-PD-1/anti-PDL1 antibody,
(ii) succinate buffer, or acetate buffer or citrate buffer, it's derivatives or salts or combinations thereof,
(iii) one or more stabilizers;
(iv) a surfactant; and
   wherein the formulation is free of a/any chelator or chelating agent.

In another embodiment, the invention discloses a method of controlling particle formation in an anti-PD-1/PD-L1 antibody composition wherein the method comprises addition of a buffer composition free of a chelator and comprising succinate buffer or acetate buffer or citrate buffer or it's derivatives or salts or combination thereof, to the antibody composition during pre-formulation and/or formulation stage of the antibody production.

In yet another embodiment, the invention discloses a method of controlling formation of charge variants in an anti-PD1/PD-L1 antibody composition wherein the method comprises addition of a buffer composition free of a chelator and comprising succinate buffer or acetate buffer or citrate buffer or it's derivatives or salts or combination thereof to the antibody composition during pre-formulation and/or formulation stage of the antibody production,

In an embodiment, the invention discloses a method of controlling aggregation and/or fragmentation of an anti-PD1/PD-L1 antibody composition wherein the method comprises addition of a buffer composition free of a chelator and comprising succinate buffer or acetate buffer o citrate buffer or it's derivatives or salts or combination thereof to the antibody composition during pre-formulation and/or formulation stage of the antibody production.

In any of the above mentioned embodiments, the succinate buffer or it's derivatives or salts or combination thereof, is a succinate buffer, or a succinate-arginine buffer or a succinate-phosphate buffer. The succinate buffer composition may also further contain at least one or more pharmaceutically acceptable excipients/stabilizer but, the buffer composition does not include any chelator or chelating agent. The said composition can be added during pre-formulation and/or formulation stage of the antibody production.

In any of the above mentioned embodiments, the citrate buffer or it's derivative or salts or combinations thereof, is a citrate buffer or a citrate-histidine buffer or a citrate-arginine or a citrate-phosphate buffer. The citrate buffer composition may also further contain at least one pharmaceutically acceptable excipients/stabilizer but does not include any chelator or chelating agent. The said composition can be added during pre-formulation and/or formulation stage of the antibody production.

In any of the above mentioned embodiments, the acetate buffer composition is an acetate buffer or an acetate-arginine buffer or an acetate-phosphate buffer. The acetate buffer composition may also further contain at least one pharmaceutically acceptable excipients/stabilizer but does not include any chelator/chelating agent. The said composition can be added during pre-formulation and formulation stage of the antibody production.

In any of the above mentioned embodiments, the one or more pharmaceutically acceptable excipients/stabilizers include sugar, polyol, or amino acid, or salt. The one or more stabilizers do not include a chelator or a chelating agent.

In an embodiment, the invention discloses a pharmaceutical formulation of an anti-PD1 antibody/anti-PD L1 antibody comprising:
i) an anti-PD1/anti-PD L1 antibody,
ii) 10-50 mM succinate buffer or acetate buffer or citrate buffer
iii) one or more stabilizers comprise mannitol, or trehalose, or sucrose or sorbitol; sodium chloride and;
iv) a surfactant, and,

wherein the formulation does not contain a chelator/chelating agent.

In any of the above mentioned embodiments, the stabilizer may include an amino acid.

In the above mentioned embodiment, the amino acid does not include methionine.

In an embodiment, the invention discloses, a liquid pharmaceutical formulation of an anti-PD1 antibody comprising, an anti-PD1 antibody, succinate or acetate buffer having a pH of 5.0 to 6.0, sugar, amino acid, and surfactant, wherein the formulation is free of chelating agent and anti-oxidant.

In any of the above mentioned embodiments, the sugar is trehalose or sucrose. In any of the above mentioned embodiment, the amino acid is glycine or proline.

In any of the above mentioned embodiments, the surfactant is polysorbate-80 or polysorbate-20.

In any of the above mentioned embodiments, the pH of the disclosed formulation of the present invention is in the range from about 4.5 to about 6.5.

In any of the above mentioned embodiments, the pH of the disclosed formulation of the present invention is in the range from about 5.0 to about 6.0.

In any of the above mentioned embodiments, the pH of the disclosed formulation of the present invention is 6.0±0.2.

In any of the above mentioned embodiments, the claimed anti-PD1-anti-PDL1 antibody formulations of the invention exhibit stability under at least one of the following conditions, wherein the temperature range from 25° C. to 50° C. for a period of time which includes from 1 day to 6 months.

In any of the above mentioned embodiments, the anti-PD1 antibody is nivolumab, pembrolizumab, cemiplimab or dosrtalimab.

In any of the above mentioned embodiment, the anti-PDL1 antibody is atezolizumab, avelumab or durvalumab.

In an embodiment the invention discloses, a liquid pharmaceutical formulation of pembrolizumab comprising pembrolizumab, 10-15 mM of acetate buffer or succinate buffer it's derivatives or salts or combination thereof having pH of 5.0 to 6.0, 4% to 8% (w/v) trehalose, 100-200 mM glycine or proline, and 0.2 mg/ml surfactant, wherein the formulation is free of chelating agent and anti-oxidant and the antibody concentration present in the formulation is in a range of 10 mg/ml to 200 mg/ml.

In an embodiment the invention discloses, a liquid pharmaceutical formulation of nivolumab antibody comprising nivolumab, 10-20 mM of acetate buffer or succinate buffer it's derivatives or salts or combination thereof, having pH of 5.0 to 6.0, 4% to 8% (w/v) trehalose, and 0.2 mg/ml surfactant, wherein the formulation is free of chelating agent or anti-oxidant and the antibody concentration present in the formulation is in a range of 10 mg/ml to 200 mg/ml.

In the above mentioned embodiment, the surfactant is polysorbate-20 or polysorbate-80.

In all of the above mentioned embodiments of the invention, the concentration of the antibody in the formulation is about 10 mg/ml to about 200 mg/ml. Preferably, the concentration of the antibody in the formulation is 10 mg/ml, or 25 mg/ml, or 30 mg/ml or 40 mg/ml or 50 mg/ml, or 60 mg/ml, or 70 mg/ml, or 80 mg/ml, 90 mg/ml, or 100 mg/ml, or 110 mg/ml, or 120 mg/ml, or 140 mg/ml or 150 mg/ml or 160 mg/ml, or 170 mg/ml or 175 mg/ml or 180 mg/ml or 190 mg/ml or 195 mg/ml or 200 mg/ml.

In any of the above said embodiments of the invention, the anti-PD1/PD-L1 antibody formulation is stable and contains less than 0.9% of high molecular weight (HMW) species or fragments in the formulation, even after storage at 40° C. for two weeks.

In any of the above mentioned embodiments, the anti-PD1/PDL1 antibody formulation's osmolality is less than 600 mOsm/kg, preferably less than 300 mOsm/kg.

The anti-PD1/PDL1 antibody formulations disclosed in the invention are biologically active.

In any of the above mentioned embodiments, the formulation of anti-PD1/PD L1 antibody is a stable liquid (aqueous) formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In any of the above embodiments of the invention, the stable liquid/aqueous formulation is suitable and can be lyophilized as lyophilized powders. Further, the lyophilized formulation of anti-PD1/PDL1 antibody can be reconstituted with appropriate diluent to achieve the liquid formulation suitable for administration.

In any of the above mentioned embodiments, the liquid/aqueous anti-PD1/PD-L1 antibody are compatible with lyophilization process and the lyophilization process does not impact quality attributes of the antibody.

Another aspect of the invention provides a vial, pre-filled syringe or autoinjector device, or any other suitable device comprising any of the subject formulations described herein. In certain embodiments, the aqueous formulation stored in the vial or pre-filled syringe or an auto injector device comprising anti-PD1/anti-PDL1 antibody, succinate buffer or acetate buffer or citrate buffer or histidine buffer or it's derivatives or combination thereof, sugar and surfactant.

Certain specific aspects and embodiments of the invention are more fully described by reference to the following examples. However, these examples should not be construed as limiting the scope of the invention in any manner.

### EXAMPLES

An anti-PD1 antibody, nivolumab, suitable for storage in the present pharmaceutical composition is produced by standard methods known in the art. For example, nivolumab is prepared by recombinant expression of immunoglobulin light and heavy chain genes in a mammalian host cell such as Chinese Hamster Ovary cells. Further, the expressed nivolumab is harvested and the crude harvest is subjected to standard downstream process steps that include purification, filtration and optionally dilution or concentration steps. For example, the crude harvest of nivolumab may be purified using standard chromatography techniques such as affinity chromatography, ion-exchange chromatography and combinations thereof. The purified nivolumab solution can additionally be subjected to one or more filtration steps, and the solution obtained is subjected to further formulation studies.

### Example 1: Assessment of Effect of Various Buffers and Stabilizers on Stability of Nivolumab Formulations

Purified nivolumab antibody approximately 25 mg/ml in various buffer back grounds such as in histidine/succinate/citrate/acetate buffer back ground was obtained from downstream chromatographic steps. To know, the effect of various buffers and/or stabilizers such as sugar/polyol/amino acid/chelators on the stability of nivolumab buffer exchange step was performed and the concentration was adjusted to 10 mg/ml. Post which, surfactant polysorbate-80 has been added to all the formulation. Nivolumab is approved under trade name Opdivo® and approved formulation contains 10 mg/ml nivolumab in 20 mM citrate buffer, 3% Mannitol, 2.92 mg/mL NaCl, 0.2 mg/mL PS 80 and 0.008 mM DTPA citric acid. Opdivo® formulation has been used in this experiment and denoted as N1. To assess the effect of chelating agent such as EDTA on nivolumab stability, EDTA was added to one of the nivolumab formulation. The final composition of all nivolumab formulations are given in Table 1. All formulations were subjected for accelerated stability study at 40° C. for 4 weeks, and at 25° C. for 4 weeks.

All the samples were measured for their particle formation, opalescence, high molecular weight species using size exclusion chromatography. To measure opalescence, various USP reference opalescence standards were prepared by diluting primary opalescence solution comprising formazin suspension having 4000 NTU ((Nephelometric Turbidity Units). All nivolumab formulations were subjected to accelerated stability studies at 40° C. for four weeks and at 25° C. for 4 weeks. The sample which were subjected for accelerated stability conditions at 25° C. for 4 weeks were further subjected for agitation at 300 RPM using an orbital shaker for three days. Post which, the samples were analyzed for high molecular weight (HMW) species and monomer content using size exclusion chromatography (SEC) [results are given in Table 2], charge variants are measured using ion-exchange chromatography (IEX) [results are given in Table 3(a) and 3(b)] and also checked for particle formation [Table 4], and opalescence [Table 5].

TABLE 1
Compositions of various nivolumab formulations
prepared as per Example-1

| Sample | |
|---|---|
| Name | Composition |
| N1 | 10 mg/ml nivolumab, 20 mM citrate buffer, 3% mannitol, |
| | 2.92 mg/mL NaCl, 0.2 mg/mL polysorbate 80 and 0.008 |
| | mM DTPA citric acid, pH 6.0. |
| N2 | 10 mg/ml nivolumab, 20 mM succinate phosphate buffer, |
| | 3% Mannitol, 2.92 mg/mL NaCl, 0.2 mg/mL polysorbate |
| | 80, pH 5.8 |
| N3 | 10 mg/ml nivolumab, 20 mM arginine-succinate buffer, |
| | 3% mannitol, 2.92 mg/mL NaCl, 0.2 mg/mL polysorbate |
| | 80, pH 5.8 |
| N4 | 10 mg/ml nivolumab, 20 mM acetate- phosphate, 3% |
| | mannitol, 50 mM NaCl, 0.2 mg/mL polysorbate-80, pH 6.1 |
| N5 | 10 mg/ml nivolumab, 20 mM succinate buffer, 3% mannitol, |
| | 50 mM mg/mL NaCl, 0.2 mg/mL polysorbate 80, pH 5.9 |
| N6 | 10 mg/ml nivolumab, 20 mM succinate buffer, 6% trehalose, |
| | 50 mM mg/mL NaCl, 0.2 mg/mL polysorbate 80, pH 5.9 |
| N7 | 10 mg/ml nivolumab, 20 mM succinate buffer, 3% sorbitol, |
| | 50 mM NaCl, 0.2 mg/mL polysorbate 80, pH 5.9 |
| N8 | 10 mg/ml nivolumab, 20 mM succinate buffer, 6% sucrose, |
| | 50 mM mg/mL NaCl, 0.2 mg/mL polysorbate 80, pH 5.9 |
| N9 | 10 mg/ml nivolumab, 20 mM succinate buffer, 3% mannitol, |
| | 0.2 mg/mL polysorbate 80, pH 6.0 |
| N10 | 10 mg/ml nivolumab, 20 mM histidine-citrate buffer, 3% |
| | mannitol, 50 mM mg/mL NaCl, 0.2 mg/mL polysorbate 80, |
| | pH 5.9 |
| N11 | 10 mg/ml nivolumab, 20 mM arginine-citrate buffer, 3% |
| | mannitol, 50 mM NaCl, 0.2 mg/mL polysorbate 80, pH 6.0 |
| N12 | 10 mg/ml nivolumab, 20 mM citrate-phosphate buffer, 3% |
| | mannitol, 50 mM NaCl, 0.2 mg/mL polysorbate 80, pH 6.0 |
| N13 | 10 mg/ml nivolumab, 20 mM citrate buffer, 3% mannitol, |
| | 50 mM NaCl, 0.2 mg/mL polysorbate 80, pH 6.0 |
| N14 | 10 mg/ml nivolumab, 20 mM succinate buffer, 3% mannitol, |
| | 50 mM NaCl, 0.05 mg/ml EDTA, 0.2 mg/mL polysorbate 80, |
| | pH 5.9 |
| N15 | 10 mg/ml nivolumab, 20 mM histidine buffer, 3% mannitol, |
| | 50 mM NaCl, 0.2 mg/mL polysorbate 80, pH 6.0 |
| N16 | 10 mg/ml nivolumab, 20 mM succinate buffer, methionine, |
| | 3% mannitol, 50 mM NaCl, 0.2 mg/mL polysorbate 80, pH 6.0 |

TABLE 2
SEC data of nivolumab formulations prepared as per
example-1 when stored at 40° C. for four weeks.

| | SEC data at 40° C. | | | | | | | |
|---|---|---|---|---|---|---|---|---|
| Sample | % Monomer at 40° C. | | | | % HMW at 40° C. | | | |
| name | T0 | T1W | T2W | T4W | T0 | T1W | T2W | T4W |
| N1 | 99.3 | 99.2 | 99.2 | 98.9 | 0.6 | 0.7 | 0.7 | 0.9 |
| N2 | 99.6 | 99.3 | 99.2 | 98.9 | 0.4 | 0.6 | 0.8 | 1 |
| N3 | 99.6 | 99.5 | 99.3 | 98.7 | 0.4 | 0.5 | 0.7 | 1.2 |
| N4 | 99.5 | 99.2 | 99.1 | 98.8 | 0.5 | 0.8 | 0.9 | 1.1 |
| N5 | 99.6 | 99.3 | 99.1 | 98.9 | 0.4 | 0.7 | 0.9 | 1 |
| N6 | 99.6 | 99.3 | 99.1 | 98.9 | 0.4 | 0.7 | 0.8 | 1 |
| N7 | 99.6 | 99.3 | 99.1 | 98.7 | 0.4 | 0.7 | 0.9 | 1.1 |
| N8 | 99.6 | 99.3 | 99.2 | 98.9 | 0.4 | 0.6 | 0.8 | 1 |
| N9 | 99.6 | 99.3 | 99.1 | 98.8 | 0.5 | 0.7 | 0.8 | 1.1 |
| N10 | 99.7 | 99.5 | 99.4 | 99.2 | 0.3 | 0.4 | 0.5 | 0.6 |
| N11 | 99.6 | 99.4 | 99.7 | 99.1 | 0.4 | 0.5 | 0.7 | 0.8 |
| N12 | 99.5 | 99.3 | 99.2 | 98.9 | 0.5 | 0.6 | 0.8 | 1 |
| N13 | 99.6 | 99.3 | 99.2 | 98.9 | 0.4 | 0.7 | 0.8 | 1 |
| N14 | 99.6 | 99.4 | 99.2 | 99.1 | 0.4 | 0.6 | 0.7 | 0.8 |
| N15 | 97.4 | 98.8 | 98.1 | 96.6 | 2.6 | 1.2 | 1.8 | 3.2 |
| N16 | 99.6 | 99.4 | 99.3 | 99.1 | 0.4 | 0.5 | 0.6 | 0.8 |

W—indicates weeks;
ND—not detected

TABLE 3 (a)
IEX data of nivolumab formulations prepared as per example-1,
when formulations stored at 40° C. for 4 weeks.

| | IEX data at 40° C. | | | | | | | |
|---|---|---|---|---|---|---|---|---|
| Sample | % Main peak content | | | | % acidic variants | | | |
| name | T0 | T1W | T2W | T4W | T0 | T1W | T2W | T4W |
| N1 | 62.8 | 60.1 | 56.0 | 47.4 | 24.3 | 26.7 | 32.0 | 37.7 |
| N2 | 57.3 | 59.1 | 55.2 | 46.8 | 19.4 | 22.2 | 27.6 | 34.2 |
| N3 | 57.6 | 57.1 | 53.9 | 47.3 | 18.9 | 20.4 | 25.6 | 32 |
| N4 | 57.3 | 59.8 | 55.7 | 48.1 | 19.5 | 22.3 | 27.8 | 33.8 |
| N5 | 57.2 | 57.3 | 54.4 | 47.4 | 19.0 | 21.9 | 27.0 | 33.3 |
| N6 | 57.4 | 57.5 | 54.3 | 47.3 | 19.0 | 21.6 | 26.8 | 33.3 |
| N7 | 57.5 | 56.8 | 53.9 | 46.9 | 19.0 | 21.8 | 27.5 | 34.4 |
| N8 | 57.5 | 57.3 | 54.7 | 47.2 | 18.8 | 21.1 | 26.3 | 32.7 |
| N9 | 57.3 | 57.5 | 54.8 | 47.1 | 19.0 | 21.6 | 27.3 | 33.8 |
| N10 | 57.2 | 56.7 | 53.8 | 47.1 | 19.2 | 21.5 | 26.8 | 32.9 |
| N11 | 57.4 | 57.5 | 54.3 | 48.2 | 18.9 | 20.3 | 25.4 | 31.4 |
| N12 | 57.9 | 59.8 | 56.1 | 48.4 | 19.1 | 22.7 | 27.6 | 33.4 |
| N13 | 58.1 | 57.7 | 54.9 | 48.3 | 18.6 | 22.1 | 27.1 | 33 |
| N14 | 57.1 | 57.2 | 55.0 | 47.4 | 19.3 | 21.3 | 26.6 | 32.8 |
| N15 | 58.8 | 57.1 | 53.7 | 47.5 | 18.8 | 21.5 | 26.9 | 32.9 |
| N16 | 57.3 | 59.0 | 54.9 | 45.8 | 19.1 | 21.2 | 26.5 | 33.3 |

TABLE 3 b
Basic variants of nivolumab formulations prepared as per example-1
measured by IEX, when formulations stored at 40° C. for 4 weeks.

| | |
|---|---|
| Sample | % basic variants |

| name | T 0 | T 1 W | T 2 W | T 4 W |
|---|---|---|---|---|
| N1 | 12.8 | 13.1 | 11.9 | 14.8 |
| N2 | 23.2 | 18.6 | 17.1 | 19 |
| N3 | 23.5 | 22.4 | 20.4 | 20.8 |
| N4 | 23.1 | 17.8 | 16.6 | 18.2 |
| N5 | 23.8 | 20.8 | 18.5 | 19.3 |
| N6 | 23.6 | 20.9 | 18.8 | 19.4 |
| N7 | 23.5 | 21.4 | 18.5 | 18.7 |
| N8 | 23.7 | 21.6 | 19.0 | 20.1 |
| N9 | 23.8 | 20.8 | 17.9 | 19.1 |
| N10 | 23.6 | 21.8 | 21.8 | 20 |
| N11 | 23.7 | 22.2 | 20.3 | 20.4 |
| N12 | 23.0 | 17.5 | 16.3 | 18.2 |
| N13 | 23.2 | 20.2 | 18.0 | 18.7 |
| N14 | 23.6 | 21.5 | 18.4 | 19.8 |
| N15 | 22.4 | 21.4 | 19.4 | 19.6 |
| N16 | 23.6 | 19.8 | 18.5 | 21 |

TABLE 4
Measurements of particle formation of
nivolumab formulations as per example 1

| | Visible particle count per 1.5 ml at 40° C. | | |
|---|---|---|---|
| Sample name | T 0 | T 2 W | T 4 W |
| N1 | 15 | 38 | 30 |
| N2 | 10 | 15 | 20 |
| N3 | 10 | 25 | 25 |
| N4 | 10 | 13 | 25 |
| N5 | 10 | 20 | 25 |
| N6 | 10 | 20 | 20 |
| N7 | 25 | 15 | 25 |
| N8 | 45 | 21 | 25 |
| N9 | 45 | 16 | 30 |
| N10 | 10 | 20 | 35 |
| N11 | 13 | 15 | 20 |
| N12 | 45 | >50 | 35 |
| N13 | 45 | 20 | 30 |
| N14 | 15 | 13 | 20 |
| N15 | 45 | 25 | >50 |
| N16 | 45 | 25 | 25 |

TABLE 5
Opalescence of nivolumab formulations prepared as per example 1

| | Opalescence at 40° C. | | | |
|---|---|---|---|---|
| Sample Name | 0 W | 1 W | 2 W | 4 W |
| N1 | ROS II | ROS II | ROS II | ROS II |
| N2 | ROS II | ROS II | ROS II | ROS II |
| N3 | ROS II | ROS II | ROS II | ROS II |
| N4 | ROS II | ROS II | ROS II | ROS II |
| N5 | ROS II | ROS II | ROS II | ROS II |
| N6 | ROS II-ROS III | ROS II | ROS II | ROS II |
| N7 | ROS II | ROS II | ROS II | ROS II |
| N8 | ROS II | ROS II | ROS II | ROS II |
| N9 | ROS II | ROS II | ROS II | ROS II |
| N10 | ROS II | ROS II | ROS II | ROS II |
| N11 | ROS II | ROS II | ROS II | ROS II |
| N12 | ROS II-ROS III | ROS II | ROS II | ROS II |
| N13 | ROS II | ROS II | ROS II | ROS II |
| N14 | ROS II | ROS II | ROS II | ROS II |
| N15 | ROS II | ROS II | ROS II | ROS II |
| N16 | ROS II | ROS II | ROS II | ROS II |

All the above formulations were also checked for change in pH. It was observed that there is no change in pH of the formulations even after storage for four weeks at 40° C. And, all the samples were observed to be colorless even after storage at 40° C. for four weeks. Osmolality of all the formulations were found to be less than 350 mOsm/kg. Further, all formulations were subjected for freeze/thaw stability studies and all formulations were found to be stable.

### Example 2: Stability of Other Anti-PD1 Antibody Formulations

Another anti-PD1 antibody, pembrolizumab expressed in CHO cells and the expressed antibody has been purified by techniques already known in the art. 35 mg/ml of purified pembrolizumab obtained from downstream chromatographic step, was subjected for buffer exchange step with succinate or histidine acetate buffer. In addition, pembrolizumab in acetate buffer obtained from downstream chromatographic technique maintained as it as. To all the pembrolizumab antibody samples in various buffers, combination of various excipients such as sugars, amino acid, chelating agents and surfactant were added. Concentration of amino acids used in this experiment is within a range of 100 mM to 200 mM. Composition of all pembrolizumab samples are given in below Table 6.

Post which, these samples were subjected for accelerated stability studies at 40° C. for one month and various quality attributes of the samples such as change in pH, osmolality; high molecular weight content, monomer content and low molecular weight content using SEC, and charge variants such as acidic variants, basic variants using IEX were measured. Further, these samples were checked for opalescence. Results of the study are given in Table 7-10.

TABLE 6
Compositions of pembrolizumab formulations
prepared as per example-2.

| Sample | |
|---|---|
| Name | Composition |
| P1 | 25 mg/ml pembrolizumab, 20 mM acetate buffer, sucrose |
| | and polysorbate-80 |
| P2 | 25 mg/ml pembrolizumab, 10 mM acetate buffer, 4.5% |
| | trehalose glycine, and polysorbate-80 |
| P3 | 25 mg/ml pembrolizumab, 10 mM acetate buffer, 4.5% |
| | trehalose proline, and polysorbate-80 |
| P4 | 25 mg/ml pembrolizumab, 10 mM acetate buffer, 10% |
| | trehalose and polysorbate-80 |
| P5 | 25 mg/ml pembrolizumab, 10 mM acetate buffer, 10% |
| | trehalose, DTPA and polysorbate-80 |
| P6 | 25 mg/ml pembrolizumab, 10 mM acetate buffer, 10% |
| | sorbitol and polysorbate-80 |
| P7 | 25 mg/ml pembrolizumab, 10 mM acetate buffer, 12% |
| | sucrose and polysorbate-80 |
| P8 | 25 mg/ml pembrolizumab, 10 mM histidine-acetate buffer, |
| | 10% trehalose and polysorbate-80 |
| P9 | 25 mg/ml pembrolizumab, 10 mM succinate buffer, 4.5% |
| | trehalose glycine, and polysorbate-80 |
| P10 | 25 mg/ml pembrolizumab, 10 mM succinate buffer, 4.5% |
| | trehalose proline, and polysorbate-80 |
| P11 | 25 mg/ml pembrolizumab, 10 mM succinate buffer, 10% |
| | trehalose and polysorbate-80 |
| P12 | 25 mg/ml pembrolizumab, 10 mM succinate buffer, 10% |
| | trehalose, DTPA, and polysorbate-80 |
| P13 | 25 mg/ml pembrolizumab, 10 mM succinate buffer, 10% |
| | sorbitol and polysorbate-80 |
| P14 | 25 mg/ml pembrolizumab, 10 mM succinate buffer, 12% |
| | sucrose and polysorbate-80 |

TABLE 7
Various quality attributes of pembrolizumab
formulations prepared as per Example-2.

| | | |
|---|---|---|
| Sample | pH at 40° C. | Osmolality (mOsm/Kg) at 40° C. |

| name | T 0 | T 2 W | T 0 | T 1 M |
|---|---|---|---|---|
| P1 | 6.0 | 5.7 | 254 | 265 |
| P2 | 6.0 | 5.8 | 291 | 304 |
| P3 | 6.0 | 5.9 | 261 | 266 |
| P4 | 6.0 | 5.9 | 326 | 337 |
| P5 | 5.9 | 5.8 | 320 | 337 |
| P6 | 5.9 | 5.7 | 608 | 633 |
| P7 | 5.9 | 5.8 | 461 | 485 |
| P8 | 6.0 | 5.8 | 357 | 371 |
| P9 | 5.8 | 5.6 | 281 | 286 |
| P10 | 5.8 | 5.6 | 267 | 271 |
| P11 | 6.5 | 5.6 | 306 | 315 |
| P12 | 5.8 | 5.6 | 305 | 317 |
| P13 | 5.8 | 5.6 | 641 | 663 |
| P14 | 5.8 | 5.6 | 402 | 418 |

T 0—represents data at zero time point;
M—indicates months;
W—indicates weeks

TABLE 8
SEC data of pembrolizumab formulations prepared as
per Example-2, when stored at 40° C. for one month.

| | | |
|---|---|---|
| Sample | % HMW content | % Monomer content |

| name | T 0 | T 1 M | T 0 | T 1 M |
|---|---|---|---|---|
| P1 | 0.5 | 0.7 | 99.4 | 99.1 |
| P2 | 0.6 | 0.8 | 99.4 | 99.1 |
| P3 | 0.6 | 1.0 | 99.4 | 99.1 |
| P4 | 0.6 | 0.8 | 99.4 | 98.9 |
| P5 | 0.6 | 0.6 | 99.4 | 99.1 |
| P6 | 0.6 | 0.9 | 99.4 | 98.7 |
| P7 | 0.6 | 0.8 | 99.4 | 99.0 |
| P8 | 0.6 | 0.8 | 99.4 | 99.2 |
| P9 | 0.6 | 1.0 | 99.4 | 99.1 |
| P10 | 0.6 | 0.9 | 99.4 | 98.9 |
| P11 | 0.6 | 0.8 | 99.4 | 99.0 |
| P12 | 0.6 | 0.6 | 99.4 | 99.1 |
| P13 | 0.6 | 0.9 | 99.4 | 98.8 |
| P14 | 0.6 | 0.8 | 99.3 | 99.1 |

T 0—represents data at zero time point;
M—indicates months

TABLE 9
IEX data of pembrolizumab formulations prepared as
per Example-6, when stored at 40° C. for one month.

| | % Acidic | | % Main peak | | % Basic variants | |
|---|---|---|---|---|---|---|
| Sample | variants | | content | | content | |
| name | T0 | T1M | T0 | T1M | T0 | T1M |
| P1 | 11.7 | 22.0 | 67.0 | 60.1 | 21.4 | 28.2 |
| P2 | 10.2 | 18.5 | 66.6 | 61.3 | 23.2 | 31.5 |
| P3 | 10.5 | 19.9 | 66.5 | 61.3 | 23.0 | 31.1 |
| P4 | 10.8 | 19.6 | 67.1 | 61.3 | 22.1 | 30.5 |
| P5 | 11.0 | 20.2 | 67.3 | 61.8 | 21.7 | 30.2 |
| P6 | 10.9 | 18.5 | 66.7 | 60.8 | 22.4 | 30.9 |
| P7 | 10.9 | 22.9 | 66.2 | 61.6 | 23.0 | 29.0 |
| P8 | 11.0 | 20.8 | 66.8 | 62.8 | 22.3 | 29.1 |
| P9 | 11.1 | 21.9 | 66.6 | 61 | 22.2 | 31.1 |
| P10 | 11.0 | 27.5 | 67.1 | 60.8 | 21.9 | 28.7 |
| P11 | 11.2 | 21.8 | 67.2 | 61.4 | 21.6 | 25.7 |
| P12 | 11.2 | 23.0 | 66.8 | 61.1 | 22.0 | 28.1 |
| P13 | 11.5 | 24.1 | 67.1 | 59.7 | 21.4 | 29.4 |
| P14 | 11.6 | 21.9 | 66.6 | 60.7 | 23.2 | 27.8 |

T0—represents data at zero time point;
M—indicates months

TABLE 10
Opalescence of nivolumab formulations prepared as per example 2

| Sample | Opalescence at 40° C. | | | |
|---|---|---|---|---|
| Name | 0 W | 1 W | 2 W | 4 W |

| | | |
|---|---|---|
| P1 | ROS-II-III and no visible | ROS-III-IV and no |
| | particles | visible particles |
| P2 | ROS-II-III and no visible | ROS-III-IV and no |
| | particles | visible particles |
| P3 | ROS-II-III and no visible | ROS-III-IV and small |
| | particles | fibrous particles |
| P4 | ROS-II-III and no visible | ROS-III-IV and small |
| | particles | fibrous particles |
| P5 | ROS-II-III and no visible | ROS-III-IV and no |
| | particles | visible particles |
| P6 | ROS-II-III and no visible | ROS-III-IV and small |
| | particles | fibrous particles |
| P7 | ROS-II-III and no visible | ROS-III-IV and no |
| | particles | visible particles |
| P8 | ROS-II-III and no visible | ROS-III-IV and no |
| | particles | visible particles |
| P9 | ROS-II-III and no visible | ROS-III-IV and small |
| | particles | fibrous particles observed |
| P10 | ROS-II-III and no visible | ROS-IV and no visible |
| | particles | particles |
| P11 | ROS-II-III and no visible | ROS-III-IV and small |
| | particles | fibrous particles |
| P12 | ROS-II-III and no visible | ROS-III-IV and small |
| | particles | fibrous particles |
| P13 | ROS-II-III and no visible | ROS-III-IV and no |
| | particles | visible particles |
| P14 | ROS-II-III and no visible | ROS-III-IV and no |
| | particles | visible particles |

### Example 3: High Concentration of Pembrolizumab Formulations

Pembrolizumab in acetate buffer at a concentration of 35 mg/ml was buffer exchanged with succinate buffer followed by concentrating upto 250 mg/ml using centrifugation filters/ultrafiltration. Alternatively, pembrolizumab in acetate buffer was concentrated upto 250 mg/ml using ultrafiltration. Post which, concentration of the antibody was adjusted to 140 mg/ml to 180 m/ml using formulation buffer and various excipients such as sugar, amino acids and surfactant were added to prepare high concentration pembrolizumab formulation. Further, the formulations were subjected for accelerated stability conditions at 40° C. for one week. Details of the formulation along with quality attributes are given in below Table 11(a) and Table 11(b).

TABLE 11 (a)
Composition of high concentration pembrolizumab formulation prepared
as per Example-3, and quality attributes of the formulations.

| | pH at 40° C. | | % HMW at 40° C. | | % Monomer at 40° C. | |
|---|---|---|---|---|---|---|
| Sample name | T0 | T1W | T0 | T1W | T0 | T1W |
| 141 mg/ml | 5.61 | 5.52 | 0.8 | 0.9 | 99.2 | 99.1 |
| pembrolizumab, 10 mM | | | | | | |
| succinate buffer, 4.5% | | | | | | |
| trehalose, 0.8% glycine | | | | | | |
| and 0.02% polysorbate-80 | | | | | | |
| 180 mg/ml | 5.92 | 5.83 | 0.8 | 1.1 | 99.2 | 98.9 |
| pembrolizumab, 10 mM | | | | | | |
| acetate buffer, 4.5% | | | | | | |
| trehalose, 0.8% glycine | | | | | | |
| and 0.02% polysorbate-80 | | | | | | |

TABLE 11 (b)
Composition of high concentration pembrolizumab formulation prepared
as per Example-3, and quality attributes of the formulations.

| | % Acidic variant | | % Main peak | | % Basic variants | |
|---|---|---|---|---|---|---|
| | at 40° C. | | content at 40° C. | | at 40° C. | |
| Sample name | T0 | T1W | T0 | T1W | T0 | T1W |
| 141 mg/ml | 6.2 | 8.0 | 65 | 65.4 | 28.8 | 28.6 |
| pembrolizumab, 10 mM | | | | | | |
| succinate buffer, 4.5% | | | | | | |
| trehalose, 0.8% glycine | | | | | | |
| and 0.02% polysorbate-80 | | | | | | |
| 180 mg/ml | 6.2 | 8.3 | 65 | 64.9 | 28.9 | 26.8 |
| pembrolizumab, 10 mM | | | | | | |
| acetate buffer, 4.5% | | | | | | |
| trehalose, 0.8% glycine | | | | | | |
| and 0.02% polysorbate-80 | | | | | | |

### Example 4: Assessment of Strength of Ionic Strength on Stability of Pembrolizumab Antibody Formulation

To assess role of ionic strength, 35 mg/ml pembrolizumab in acetate buffer obtained from downstream chromatographic step was formulated in either 10 mM or 20 mM acetate buffer. To which, excipients such as trehalose, arginine and surfactant were added. Post which the samples were subjected for accelerated stability studies at 40° C. for two weeks. And, high molecular weight species, monomer content and low molecular weight contents of these samples were measured using SEC chromatography. Results of the study are given in below 10 Table 12.

TABLE 12
Composition of pembrolizumab formulation prepared as per Example-4,
and size variants of the formulations measured by SEC.

| | % HMW at | | % Monomer | | % LMW | |
|---|---|---|---|---|---|---|
| | 40° C. | | at 40° C. | | at 40° C. | |
| Sample name | T0 | T2W | T0 | T2W | T0 | T2W |
| 25 mg/ml pembrolizumab, | 1.4 | 3.3 | 98.6 | 96.7 | ND | ND |
| 10 mM acetate buffer, | | | | | | |
| 4.5% trehalose, 1.3 mg/ml | | | | | | |
| arginine and 0.02% | | | | | | |
| polysorbate-80 | | | | | | |
| 25 mg/ml pembrolizumab, | 1.1 | 1.4 | 98.9 | 98.6 | ND | ND |
| 10 mM acetate buffer, | | | | | | |
| 4.5% trehalose, 1.3 mg/ml | | | | | | |
| arginine and 0.02% | | | | | | |
| polysorbate-80 | | | | | | |

ND—indicates not detected;
T0—indicates data at T0 time point

1. A liquid pharmaceutical formulation of an anti-PDI antibody comprising, an anti-PDI antibody, succinate or acetate buffer or it's derivatives or salts or combination thereof, having a pH of 5.0 to 6.0, sugar, amino acid, and surfactant, and wherein the formulation is free of chelating agent.

2. The formulation as claimed in claim 1, wherein the anti-PDI antibody concentration ranges from 10 mg/ml to 200 mg/ml.

3. The formulation as claimed in claim 1, wherein the anti-PDI antibody is nivolumab or pembrolizumab.

4. The formulation as claimed in claim 1, wherein the sugar is trehalose or sucrose.

5. The formulation as claimed in claim 1, wherein the surfactant is polysorbate 80 or polysorbate 20.

6. The formulation as claimed in claim 1, wherein the amino acid is glycine or proline.

7. The formulation as claimed in claim 6, wherein the amino acid does not include methionine.

8. A liquid pharmaceutical formulation of pembrolizumab antibody comprising, pembrolizumab, 10-15 mM of acetate buffer or succinate buffer it's derivatives or salts or combination thereof, having pH of 5.0 to 6.0, 4% to 8% (w/v) trehalose, 100-200 mM glycine or proline, and 0.2 mg/ml surfactant, wherein the formulation is free of chelating agent and anti-oxidant.

9. A liquid pharmaceutical formulation of nivolumab antibody comprising, nivolumab, 10-20 mM of succinate buffer or acetate buffer it's derivatives or salts or combination thereof, having pH of 5.0 to 6.0, 4% to 8% (w/v) trehalose, and 0.2 mg/ml surfactant, wherein the formulation is free of chelating agent.

10. The formulation as claimed in claim 1, wherein the chelating agent includes ethylenediaminetetraacetic acid (EDTA), and the formulation is devoid of EDTA.
